(19) United States
(12) Patent Application Publication
(10) Pub. No.: US 2010/0202981 A1
(43) Pub. Date: Aug. 12, 2010

(54) COMPOSITIONS HAVING IMPROVED SUBSTANTIVITY

(76) Inventor: Pauline Pan, Denville, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/765,333

(22) Filed: Apr. 22, 2010

Related U.S. Application Data
(62) Division of application No. 11/133,098, filed on May 19, 2005.

(51) Int. Cl. A61K 9/68; A61K 8/18; A61K 8/21; A61Q 11/00

(52) U.S. Cl. 424/48; 424/49; 424/52; 424/53

(57) ABSTRACT

The present invention relates to oral care compositions providing improved substantivity to and for the tissues and hard surfaces of the oral cavity.

The present invention relates to oral care compositions providing improved substantivity to and for the tissues and hard surfaces of the oral cavity.

### BACKGROUND OF THE INVENTION

The benefits of maintaining oral hygiene are well understood. Consumers understand the benefits of daily oral treatments such as brushing teeth and the use of mouth rinses. These benefits include the reduction of caries, plaque, and gingivitis; treating hypersensitivity; freshening breath; whitening teeth and removing stains; remineralizing teeth and the like. An increasing consumer desire, if not requirement, is the need to maintain their teeth for life. Consumers relate healthy oral tissues and “fresh breath” with a healthy body and lifestyle. A wide variety of oral care products have been developed to aid in the short-term maintenance of good oral hygiene. These products deliver various oral care benefit agents to the soft and hard tissues of the oral cavity in such a way that, in general, they are intended for application by the consumer themselves during part of their daily routine, and/or are administered by oral hygiene specialists in the course of administering treatment.

The most frequently used oral care treatments used in the western world are those treatments that are administered by the consumer themselves once or twice a day as part of a daily routine. Examples of such treatments include dentifrices containing for example anti-bacterial plaque actives and/or anti-caries actives and mouth rinses containing anti-bacterial actives and/or breath freshening actives. A continuing need in our society relates to oral care products capable of providing continuous 24-hour oral care maintenance. While some of the above mentioned treatments claim extended or prolonged therapeutic benefits following the initial treatment, they do not typically meet the needs of the consumer in providing substantial long lasting therapeutic, prophylactic and/or cosmetic treatment benefits. As a result, the only way to achieve sustained active release has been to periodically reapply the product, or to use special delivery mechanisms such as a dental tray. Also, despite the common acceptance and use of extended daily oral regimens such as brushing, rinsing and flossing, unsatisfactory morning mouth feel and malodor are still consumer concerns.

A need exists for improved compositions and methods for delivering oral care benefit agents to a consumer over an extended period of time without the requirement of further application or intervention following the initial application.

Additionally, a need exists for oral care products having a mouth feel which is pleasant and acceptable in the oral cavity over extended periods of time. Acceptable mouth feel is advantageous as it encourages regular consumer usage. Long-term mouth-feel is recognized as a balancing act between substantivity, adherence, and viscosity. Desirable products require sufficient substantivity and viscosity to enable application to the oral cavity, to adhere to the oral tissues and to release the contained oral care benefit agents over an extended period of time. However, the viscosity should not be so high that the consumer can feel globular portions of the newly applied product that have not spread well over the oral tissues upon application. It is desirable to have a composition that enables easy application to the oral cavity, thin layer formation over the oral tissues and even spread into periodontal pockets and fissures

### SUMMARY OF THE INVENTION

The present invention relates to oral care compositions having improved substantivity, comprising
a.) an effective amount of an oral care active; and
b.) an effective amount of a substantivity enhancing agent selected from the group consisting of linseed polysaccharide base compound, tamarind seed polysaccharide base compound, and mixtures thereof.

The present invention also relates methods of using such compositions.

### DETAILED DESCRIPTION OF THE INVENTION

As used herein the term “comprising” means that the composition can contain other ingredients which are compatible with the composition and which preferably do not substantially disrupt the compositions of the present invention. The term encompasses the terms “consisting of” and “consisting essentially of”.

Unless otherwise indicated, all percentages and ratios used herein are by weight of the total composition. All weight percentages, unless otherwise indicated, are on an actives weight basis. All measurements made are at 25° C., unless otherwise designated.

The term “oral care active” as used herein refers to any composition which has a prophylactic, therapeutic or cosmetic benefit either directly within the oral cavity or which is absorbed via the oral cavity but which has its primary benefit elsewhere. The term “treatment” as used herein refers to process of applying a substance to the oral cavity, wherein that substance may or may not comprise an oral care active, such that a prophylactic, therapeutic or cosmetic benefit is achieved.

The term “oral cavity” as referred to herein refers to the cavity from the lips to the epiglottis. The “hard tissues” comprise tissues such as the teeth and periodontal support and the like and the “soft tissues” comprise tissues such as the gums, the tongue, the surfaces of the buccal cavity and the like. Within the scope of this application the hard and soft tissues of the oral cavity should also be considered to comprise any devices which are used therein for example dentures, partial dentures, braces and the like.

The term “substantive” or “substantivity” as used herein is understood to mean that sufficient quantities of the composition and/or oral care active are retained or are capable of retention in the oral cavity such that they can be perceived by the consumer either visually or by feel after a certain time period has elapsed.

The term “effective amount” as referred to herein refers to an amount of the oral care active agent and/or the substantivity enhancing agent that is sufficient to at least reduce or relieve the condition, symptom, or disease being treated, but low enough to avoid any adverse side effects.

### Substantivity Enhancing Agent

A component of the present invention is a substantivity enhancing agent. The substantivity enhancing agent active is selected from a group consisting of or consisting essentially of linseed extracts, tamarind seed extracts, and mixtures thereof.

### Linseed Extract or Polysaccharide Base Compound

In an embodiment of the present invention, the substantivity enhancing agent relates to extracts and/or polysaccharides of the type which are present in linseed and possess rheological and surface-chemical properties which make it substantive and/or useful for improving substantivity. Certain embodiments of the present invention incorporate water soluble linseed polysaccharides.

These polysaccharides are directly obtainable from linseed by a simple extraction. One way of obtaining said polysaccharides, which will be described more in detail below, therefore is to directly dissolve the polysaccharides from linseed by means of water, but of course the invention is not limited to such an embodiment. Any polysaccharide fraction having the corresponding or essentially similar composition and obtainable in any other way, even synthetically, is useful for purposes of the present invention. Hence, alternatively, the polysaccharides can be extracted, or the major proportion thereof, from linseed by means of water in combination with other solvents, e.g. ethanol (for instance up to 70% of ethanol in water) or even completely other solvents than water, provided that said combinations or other solvents dissolve essentially the same polysaccharides as water. Conventional measures can be used to remove any excess solvent.

Certain embodiments of the present invention incorporate linseed polysaccharides in the form of an aqueous solution having a viscosity within the range of 1-30 centipoise or, optionally, 2-10 centipoise (Brookfield RVT, #5 RV Spindle, 10 rpm, 25° C.).

The concentration of the linseed polysaccharide in the oral care compositions of the present invention can range from about 0.1% to about 15% or optionally from about 1% to about 8% by weight of the oral care composition. Solutions of linseed polysaccharide are available from Sinclair Pharma under the trade name Salinum®, or from Rita Corporation under the trade name Sensiline®.

Certain embodiments of the present invention incorporate linseed extract/polysaccharide solutions containing inorganic salts (e.g., sodium chloride and potassium bicarbonate) typically present in human saliva. The total level of such salts can range up to about 3 mg per ml of water.

Additional information concerning the rheological properties of linseed extracts/polysaccharides can be found in K. Wannerberger (1990) Unconventional Sources for Food and Feed, Food Technology Series, the University of Lund, S-221 00 Lund. Further discussion of linseed extracts can be found in U.S. Pat. No. 5,260,282 to Attstrom et al., herein incorporated by reference in its entirety.

### Tamarind Seed Extract or Polysaccharide Base Compound

In an embodiment of the present invention, the substantivity enhancing agent relates to extracts and/or polysaccharides of the type which are present in tamarind seed (Tamarindus indica) and possess rheological and surface-chemical properties which make it substantive and/or useful for improving substantivity. Certain embodiments of the present invention incorporate water soluble tamarind seed polysaccharides.

These polysaccharides are directly obtainable from tamarind seed by an extraction and purification process described in U.S. Pat. No. 6,056,950, to Saettone et al., incorporated herein by reference in its entirety. Tamarind seed extracts are further discussed in U.S. Pat. No. 3,399,189, to Gordon, herein incorporated by reference in its entirety.

The concentration of the tamarind seed polysaccharide in the oral care compositions of the present invention can range from about 0.1% to about 15% or optionally from about 1% to about 8% by weight of the oral care composition. Preparations containing tamarind seed polysaccharide are commercially available under the trademark GLYLOID®, a product of Dainippon Pharmaceutical Co., Ltd.

### Oral Care Active Agents

The compositions of the present invention comprise at least one oral care active agent. Oral care active agents of the present invention may be selected from the group including anti-microbial agents, desensitizing agents, teeth whitening actives, anti-stain agents, anti-tartar agents, anti-plaque agents, fluoride ion sources, tooth strengthening agents, nutrients, antioxidants, H-2 antagonists and mixtures thereof. The oral care active agent may comprise from about 0.01% to about 15% by weight of the carrier. The following is a non exclusive list of oral care active agents that may be used in the present invention:

1. Tooth Whitening Actives
Tooth whitening actives may be included in the oral care benefit agent of the present invention. The actives suitable for whitening include, but are not limited to peroxides, metal chlorites, perborates, percarbonates, peroxyacids, and combinations thereof. Suitable peroxide compounds include hydrogen peroxide, calcium peroxide, carbamide peroxide, and mixtures thereof. Suitable metal chlorites include, but are not limited to, calcium chlorite, barium chlorite, magnesium chlorite, lithium chlorite, sodium chlorite, and potassium chlorite. Additional whitening actives may be the hypochlorite salts and chlorine dioxide. Mixtures of the above teeth whitening actives may also be used.

2. Anti-tartar agents
Anti-tartar agents known for use in dental care products include, but are not limited to, pyrophosphates, linear polyphosphates with 4 or more repeat units, polyphosphonates and mixtures thereof. Pyrophosphate ions delivered to the teeth are derived from pyrophosphate salts. The pyrophosphate salts are described in more detail in Kirk & Othmer, Encyclopedia of Chemical Technology, Third Edition, Volume 17, Wiley-Interscience Publishers (1982). Agents that may be used in place of or in combination with pyrophosphate salts include, but are not limited to, such known materials as synthetic anionic polymers including polyacrylates and copolymers of maleic anhydride or acid and methyl vinyl ether, as described, for example, in U.S. Pat. No. 4,627,977 to Gaffer et al. herein incorporated by reference; as well as, e.g., polyamino propoane sulfonic acid (AMPS), zinc citrate trihydrate, linear polyphosphates (e.g., tripolyphosphate; hexametaphosphate), diphosphonates (e.g., ethane-1-hydroxy-1,1-diphosphonate, 1-azacycloheptane-1,1-diphosphonate), polypeptides (such as polyaspartic and polyglutamic acids), and mixtures thereof. Further antitartar agents include polycarboxylates; polyepoxysuccinates; ethylenediaminetetraacetic acid; linear alkyl diphosphonates; linear carboxylic acids; sodium zinc citrate, nitrilotriacetic acid and related compounds and mixtures thereof. A more detailed discussion of suitable antitarter agents can be found in U.S. Pat. No. 6,682,722 to Majeti, herein incorporated by reference in its entirety.

3. Fluoride Ion Source
Fluoride ion sources are well known for use in oral care compositions as anticaries agents. Fluoride ions are contained in a number of oral care compositions for this purpose. A wide variety of fluoride ion-yielding materials can be employed as sources of soluble fluoride in the instant aqueous gels. Examples of suitable fluoride ion-yielding materials include, but are not limited to, sodium fluoride, stannous fluoride, sodium monofluorophosphate and mixtures thereof. In certain embodiments, the instant compositions provide from about 50 ppm to 10,000 ppm, more preferably from about 100 to 3000 ppm, of fluoride ions.

4. Antimicrobial Agents
Suitable oral care benefit agents herein also include anti-microbial agents. Antimicrobial agents are known to those skilled in the art and include, but not limited to, cationic agents, non-cationic agents and metal ion salts. Such agents may include, but, again, are not limited to, 5-chloro-2-(2,4-dichlorophenoxy)-phenol, commonly referred to as triclosan, and described in The Merck Index, 11th ed. (1989), pp. 1529 (entry no. 9573); phthalic acid and its salts, substituted monoperthalic acid and its salts and esters, optionally, magnesium monoperoxy phthalate, chlorhexidine (Merck Index, no. 2090), alexidine (Merck Index, no. 222; hexetidine (Merck Index, no. 4624); sanguinarine (Merck Index, no. 8320); benzalkonium chloride (Merck Index, no. 1066); salicylanilide (Merck Index, no. 8299); domiphen bromide (Merck Index, no. 3411); cetylpyridinium chloride (CPC) (Merck Index, no. 2024; tetradecylpyridinium chloride (TPC); N-tetradecyl-4-ethylpyridinium chloride (TDEPC); octenidine; delmopinol, octapinol, and other piperidino derivatives; nicin preparations; zinc/stannous ion agents; antibiotics such as augmentin, amoxicillin, tetracycline, doxycycline, minocycline, and metronidazole; and analogs and salts of the above; essential oils including thymol, geraniol, carvacrol, citral, hinokitiol, methyl salicylate, eucalyptol, menthol, catechol (particularly 4-allyl catechol) and mixtures thereof; hydrogen peroxide; nanochitosan, metal salts of chlorite, and mixtures of any and/or all of the above. In certain embodiments, the antimicrobial agents include cetyl pyridinium chloride, triclosan and mixtures thereof. In certain embodiments, the antimicrobial agent comprises from about 0.05% to about 3%, optionally, from about 0.1% to about 1.5% by weight of the oral care composition.

5. Anti-inflammatory Agents
Anti-inflammatory agents can also be present in the compositions of the present invention. Such agents may include, but are not limited to, non-steroidal anti-inflammatory agents (or NSAIDs) such as ketorolac, flurbiprofen, ibuprofen, naproxen, indomethacin, aspirin, ketoprofen, piroxicam and meclofenamic acid. Use of NSAIDs such as Ketorolac are claimed in U.S. Pat. No. 5,626,838 to Cavanaugh. Disclosed therein are methods of preventing and, or treating primary and reoccurring squamous cell carcinoma of the oral cavity or oropharynx by topical administration to the oral cavity or oropharynx an effective amount of an NSAID. Also, useful are cox-2 inhibitors such as celecoxib, valdecoxib, deracoxib, etoricoxib, rofecoxib, ABT-963 (2-(3,4-difluorophenyl)-4-(3-hydroxy-3-methyl-1butoxy)-5-[4-methylsulfonyl)phenyl-3(2H)-pyridazinone; described in PCT Patent Application No. WO 00/24719) herein incorporated by reference, or meloxicam and mixtures of any and/or all the above. A compound of the present invention can also be advantageously used in therapeutic combination with a prodrug of a COX-2 selective inhibitor, for example parecoxib.

6. Nutrients
Nutrients may improve the condition of the oral cavity and can be included in the compositions of the present invention. Nutrients include minerals, vitamins, nutritional supplements, and mixtures thereof.
Minerals that can be included with the compositions of the present invention include, but are not limited to, calcium, phosphorus, fluoride, zinc, manganese, potassium and mixtures thereof. These minerals are disclosed in Drug Facts and Comparisons (loose leaf drug information service), Wolters Kluer Company, St. Louis, Mo., &copy; 1997, pp 10.17.
Vitamins can be included with minerals or used separately. Vitamins include Vitamins C and D; thiamine; riboflavin; calcium pantothenate; niacin; folic acid; nicotinamide; pyridoxine; cyanocobalamin; para-aminobenzoic acid; bioflavonoids; and mixtures thereof. Such vitamins are disclosed in Drug Facts and Comparisons (loose leaf drug information service), Wolters Kluer Company, St. Louis, Mo., &copy; 1997, pp. 3-10.
Nutritional supplements include amino acids, lipotropics, fish oil, protein products, glucose polymers, corn oil, safflower oil, medium chain triglycerides and mixtures thereof, as disclosed in Drug Facts and Comparisons (loose leaf drug information service), Wolters Kluer Company, St. Louis, Mo., &copy; 1997, pp. 54-54e. Amino acids include, but, are not limited to L-Tryptophan, L-Lysine, Methionine, Threonine, Levocarnitine or L-carnitine and mixtures thereof. Lipotropics include, but are not limited to choline, inositol, betaine, linoleic acid, linolenic acid, and mixtures thereof. Fish oil contains large amounts of Omega-3 (N-3) polyunsaturated fatty acids, eicosapentaenoic acid and docosahexaenoic acid.

7. Enzymes
An individual or combination of several compatible enzymes can be included in the compositions of the present invention. Enzymes are biological catalysts of chemical reactions in living systems. Enzymes combine with the substrates on which they act forming an intermediate enzyme-substrate complex. This complex is then converted to a reaction product and a liberated enzyme which continues its specific enzymatic function.
Enzymes provide several benefits when used in the oral cavity. Proteases break down salivary proteins which are absorbed onto the tooth surface and form the pellicle; the first layer of plaque. Proteases along with lipases destroy bacteria by lysing proteins and lipids which form the structural component of bacterial cell walls and membranes. Dextranases break down the organic skeletal structure produced by bacteria that forms a matrix for bacterial adhesion. Proteases and amylases, not only present plaque formation, but also prevent the development of calculus by breaking up the carbohydrate-protein complex that binds calcium, preventing mineralization.
Enzymes useful in the present invention include, but are not limited to, any of the commercially available proteases, glucanohydrolases, endoglycosidases, amylases, mutanases, lipases and mucinases or compatible mixtures thereof. Certain embodiments of the present invention incorporate proteases, dextranases, endoglycosidases and mutanases. Other embodiments incorporate papain, endoglycosidase or a mixture of dextranase and mutanase.

8. Antioxidants
Antioxidants are generally recognized as useful in the compositions of the present invention. Antioxidants are disclosed in texts such as Cadenas and Packer, The Handbook of Antioxidants, &copy; 1996 by Marcel Dekker, Inc. Antioxidants that may be included in the compositions of the present invention include, but are not limited to, Vitamin E, ascorbic acid, Uric acid, carotenoids, Vitamin A, avenanthramide, flavonoids and polyphenols, herbal antioxidants, melatonin, aminoindoles, lipoic acids and mixtures thereof.

9. Anesthetics
Local anesthetic agents suitable for use in the practice of this invention include amides and esters. Examples of the amides are lidocaine, prilocaine, mepivacaine, bupivacaine, dibucaine and etidocaine. Esters include procaine, tetracaine, propoxycaine, chloroprocaine, benzocaine, butamben picrate, cocaine, hexylcaine, piperocaine, oxyprocaine and proparacaine. Other suitable local anesthetics for use in the practice of this invention include cyclomethycaine, dimethisoquin, ketocaine, diperodon, dyclonine and pramoxine, all typically administered in the form of the acid addition hydrochloride or sulfate salts.
The acid-addition salts of anesthetic agents suitable for the present invention include any non-toxic, pharmaceutically acceptable organic or inorganic salts which in certain embodiments are non-salicylate. Typical inorganic salts are the hydrogen halides, especially the hydrochlorides, carbonates, borates, phosphates, sulfates, hydrogen sulfates, hydrobromides, nitrates, sulfides, and arsenates. Typical organic salts are salts of mono- and polycarboxylic acids such as the citrate, tartrate, malate, cinnamate, oxalate, formate, succinate and phthalates. The base form and the salt form of a suitable anesthetic agent incorporated in the present composition should preferably be different anesthetic agents to achieve maximum duration of the combined anesthetic effect. The term “different” when used with reference to an anesthetic agent means that the salt form in any combination is not a salt of the base form used in the given combination.

In addition to the components described above, the present compositions may comprise additional components, which are described in the following paragraphs

### Orally Acceptable Carrier

The orally acceptable carrier comprises one or more compatible solid or liquid filler diluents or encapsulating substances which are suitable for topical oral administration. By “compatible,” as used herein, is meant that the components of the composition are capable of being commingled without interaction in a manner which would substantially reduce the composition's stability and/or efficacy.

The carriers or excipients of the present invention can include the usual and conventional components of dentifrices (including non-abrasive gels and gels for subgingival application), mouth rinses, mouth sprays, chewing gums, chewable tablets, chewy confectioneries, edible and/or bio-adhesive films and lozenges (including breath mints) as more fully described hereinafter.

The choice of a carrier to be used is basically determined by the way the composition is to be introduced into the oral cavity. If a toothpaste (including tooth gels, etc.) is to be used, then a “toothpaste carrier” is chosen (e.g., abrasive materials, sudsing agents, binders, humectants, flavoring and sweetening agents, etc.) as disclosed in, e.g., U.S. Pat. No. 3,988,433, to Benedict, herein incorporated by reference. If a mouth rinse is to be used, then a “mouth rinse carrier” is chosen (e.g., water, flavoring and sweetening agents, etc.), as disclosed in, e.g., U.S. Pat. No. 3,988,433 to Benedict. Similarly, if a mouth spray is to be used, then a “mouth spray carrier” is chosen or if a lozenge is to be used, then a “lozenge carrier” is chosen (e.g., a candy base), candy bases being disclosed in, e.g., U.S. Pat. No. 4,083,955 to Grabenstetter et al., herein incorporated by reference; if a chewing gum is to be used, then a “chewing gum carrier” is chosen (e.g., gum base, flavoring and sweetening agents), as disclosed in, e.g., U.S. Pat. No. 4,083,955, to Grabenstetter et al. If a sachet is to be used, then a “sachet carrier” is chosen (e.g., sachet bag, flavoring and sweetening agents). If a subgingival gel is to be used (for delivery of actives into the periodontal pockets or around the periodontal pockets), then a “subgingival gel carrier” is chosen as disclosed in, e.g. U.S. Pat. Nos. 5,198,220 and 5,242,910, and respectively both to Damani, both of which are herein incorporated by reference. Carriers suitable for the preparation of compositions of the present invention are well known in the art. Their selection will depend on secondary considerations like taste, cost, and shelf stability, etc.

The compositions of the present invention may be in the form of non-abrasive gels, including subgingival gels, which may be aqueous or non-aqueous. Aqueous gels generally include a thickening agent (from about 0.1% to about 20%), a humectant (from about 10% to about 55%), a flavoring agent (from about 0.04% to about 2%), a sweetening agent (from about 0.1% to about 3%), a coloring agent (from about 0.01% to about 0.5%), and the balance water. The compositions may comprise an anticaries agent (from about 0.05% to about 0.3% as fluoride ion), and an anticalculus agent (from about 0.1% to about 13%).

In certain embodiments, the compositions of the subject invention may also be in the form of dentifrices, such as toothpastes, tooth gels and tooth powders. Components of such toothpaste and tooth gels generally include one or more of a dental abrasive (from about 6% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an anticaries agent (from about 0.05% to about 0.3% as fluoride ion), and an anticalculus agent (from about 0.1% to about 13%). Tooth powders, of course, contain substantially all non-liquid components.

Other embodiments are in the form of mouthwashes, including mouth sprays. Components of such mouthwashes and mouth sprays typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of an anticaries agent (from about 0.05% to about 0.3% as fluoride ion), and an anticalculus agent (from about 0.1% to about 3%).

Still other embodiments are in the form of dental solutions including irrigation fluids. Components of such dental solutions generally include one or more of water (from about 90% to about 99%), preservative (from about 0.01% to about 0.5%), thickening agent (from 0% to about 5%), flavoring agent (from about 0.04% to about 2%), sweetening agent (from about 0.1% to about 3%), and surfactant (from 0% to about 5%).

Chewing gum composition embodiments typically include one or more of a gum base (from about 50% to about 99%), a flavoring agent (from about 0.4% to about 2%) and a sweetening agent (from about 0.01% to about 20%).

The term “lozenge” as used herein includes: breath mints, troches, pastilles, microcapsules, and fast-dissolving solid forms including freeze dried forms (cakes, wafers, thin films, tablets) and fast-dissolving solid forms including compressed tablets. The term “fast-dissolving solid form” as used herein means that the solid dosage form dissolves in less than about 60 seconds, preferably less than about 15 seconds, more preferably less than about 5 seconds, after placing the solid dosage form in the oral cavity. Fast-dissolving solid forms are disclosed in U.S. Pat. Nos. 4,642,903 to Davies, 4,946,684 to Blank et al., 4,305,502 to Bredal, 4,371,516 to Gregory et al., 5,188,825 to Iles et al., 5,215,756 to Gole et al., 5,298,261 to Pebly et al., 3,882,228 to Boncey et al., 4,687,662 to Schobel, each of which are herein incorporated by reference.

Lozenges include discoid-shaped solids comprising a therapeutic agent in a flavored base. The base may be a hard sugar candy, glycerinated gelatin or combination of sugar with sufficient mucilage to give it form. These dosage forms are generally described in Remington: The Science and Practice of Pharmacy, 19 (th) Ed., Vol. 11, Chapter 92, 1995. Lozenge compositions (compressed tablet type) typically include one or more fillers (compressible sugar), flavoring agents, and lubricants. Microcapsules of the type contemplated herein are disclosed in U.S. Pat. No. 5,370,864, Peterson et al.

Edible or bioadhesive film embodiments include, but are not limited to, such film embodiments as that described in U.S. Pat. Nos. 6,596,298 to Leung et al., 6,419,903 to Xu et al., and 6,656,493 to Dzija et al., each of which is herein incorporated by reference in its entirety.

Types of carriers or oral care excipients which may be included in compositions of the present invention, along with specific non-limiting examples, are discussed in the following paragraphs.

### Abrasives

Dental abrasives useful in the topical, oral carriers of the compositions of the subject invention include many different materials. The material selected must be one which is compatible within the composition of interest and does not excessively abrade dentin. Suitable abrasives include, but are not limit to, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde. Another class of abrasives for use in the present compositions is the particulate thermo-setting polymerized resins as described in U.S. Pat. No. 3,070,510 to Cooley & Grabenstetter, herein incorporated by reference. Suitable resins include, for example, melamines, phenolics, ureas, melamine-ureas, melamine-formaldehydes, urea-formaldehyde, melamine-urea-formaldehydes, cross-linked epoxides, and cross-linked polyesters. Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, and DiGiulio, U.S. Pat. No. 3,862,307, each of which are herein incorporated by reference. Certain embodiments incorporate the silica xerogels marketed under the trade name “Syloid” by the W. R. Grace & Company, Davison Chemical Division. Other embodiments incorporate precipitated silica materials such as those marketed by the J. M. Huber Corporation under the trade name, Zeodent®, particularly the silicas carrying the designation Zeodent® 119, Zeodent® 118, Zeodent® 109 and Zeodent® 129. The types of silica dental abrasives useful in the toothpastes of the present invention are further described in more detail in Wason, U.S. Pat. No. 4,340,583, and in commonly-assigned U.S. Pat. No. 5,603,920, U.S. Pat. No. 5,589,160, U.S. Pat. No. 5,658,553, and U.S. Pat. No. 5,651,958, to Rice, each of which are herein incorporated by reference.

Mixtures of the above abrasives can also be used such as mixtures of the various grades of Zeodent® silica abrasives listed above. The total amount of abrasive in dentifrice compositions of the subject invention can, optionally, range from about 6% to about 70% by weight; toothpastes optionally contain from about 10% to about 50% of abrasives, by weight of the composition. In certain solution, mouth spray, mouthwash and non-abrasive gel composition embodiments of the subject invention, abrasives are typically not present.

### Surfactants

The present compositions may also comprise surfactants, also commonly referred to as sudsing or detergent agents. Suitable surfactants are those which are reasonably stable and foam throughout a wide pH range. The surfactant may be anionic, nonionic, amphoteric, zwitterionic, cationic, or mixtures thereof.

Anionic surfactants useful herein include, but are not limited to, the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458, herein incorporated by reference. The present composition typically comprises an anionic surfactant at a level of from about 0.025% to about 9%, optionally from about 0.05% to about 5%, or, optionally, from about 0.1% to about 1%.

Certain embodiments of the present invention contain surfactants selected from the group consisting of sarcosinate surfactants, isethionate surfactants, taurate surfactants and mixtures thereof. Alkali metal or ammonium salts of these surfactants may optionally be used. Some embodiments of the present invention incorporate sodium and potassium salts of the following: lauroyl sarcosinate, myristoyl sarcosinate, palmitoyl sarcosinate, stearoyl sarcosinate and oleoyl sarcosinate. The surfactant can be present in the compositions of the present invention at from about 0.1% to about 2.5%, optionally, from about 0.3% to about 2.5% or, optionally, from about 0.5% to about 2.0% by weight of the total composition.

Useful cationic surfactants are broadly defined as derivatives of aliphatic quaternary ammonium compounds having one long alkyl chain containing from about 8 to 18 carbon atoms such as lauryl trimethylammonium chloride; cetyl pyridinium chloride; cetyl trimethylammonium bromide; di-isobutylphenoxyethyl-dimethylbenzylammonium chloride; coconut alkyltrimethylammonium nitrite; cetyl pyridinium fluoride; etc. Suitable compounds are the quaternary ammonium fluorides described in U.S. Pat. No. 3,535,421, Oct. 20, 1970, to Briner et al., herein incorporated by reference, where said (quaternary ammonium fluorides have detergent properties. Certain cationic surfactants can also act as germicides in the compositions disclosed herein. Cationic surfactants such as chlorhexidine, although suitable for use in the current invention, are not contained in certain embodiments.

Nonionic surfactants suitable for use in the compositions of the present invention can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Zwitterionic synthetic surfactants useful in the present invention can be broadly described as derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate. Suitable betaine surfactants are disclosed in U.S. Pat. No. 5,180,577 to Polefka et al., herein incorporated by reference. Typical alkyl dimethyl betaines include, but are not limited to, decyl betaine or 2-(N-decyl-N,N-dimethylammonio)acetate, coco betaine or 2-(N-coc-N,N-dimethyl ammonio) acetate, myristyl betaine, palmityl betaine, lauryl betaine, cetyl betaine, cetyl betaine, stearyl betaine, etc and mixtures thereof. The amidobetaines are exemplified by cocoamidoethyl betaine, cocoamidopropyl betaine, lauramidopropyl betaine and the like and mixtures thereof. Certain embodiments of the present invention incorporate cocoamidopropyl betaine or lauramidopropyl betaine or mixtures thereof

### Chelating Agents

Another optionally useful agent is a chelating agent such as tartaric acid and pharmaceutically-acceptable salts thereof, citric acid and alkali metal citrates and mixtures thereof.

Certain embodiments incorporate sodium and/or potassium citrate as the alkali metal citrates. Also useful is a citric acid/alkali metal citrate combination. Other embodiments incorporate alkali metal salts of tartaric acid. Suitable for use herein are disodium tartrate, dipotassium tartrate, sodium potassium tartrate, sodium hydrogen tartrate, potassium hydrogen tartrate and mixtures thereof. The amounts of chelating agent suitable for use in the present invention are about 0.1% to about 2.5%, preferably from about 0.5% to about 2.5% and more preferably from about 1.0% to about 2.5%. The tartaric acid salt chelating agent can be used alone or in combination with other optional chelating agents.

Other optional chelating agents can be used. These chelating agents can have a calcium binding constant of about 10 (1) to 10 (5) provide improved cleaning with reduced plaque and calculus formation.

Still another possible group of chelating agents suitable for use in the present invention are the anionic polymeric polycarboxylates. Such materials are well known in the art, being employed in the form of their free acids or partially or fully neutralized water soluble alkali metal (e.g. potassium and preferably sodium) or ammonium salts. Useful herein are 1:4 to 4:1 copolymers of maleic anhydride or acid with another polymerizable ethylenically unsaturated monomer, preferably methyl vinyl ether (methoxyethylene) having a molecular weight (M.W.) of about 30,000 to about 1,000,000. These copolymers are available, for example, as Gantrez AN 139 (M.W. 500,000), AN 119 (M.W. 250,000) and preferably S-97 Pharmaceutical Grade (M.W. 70,000), of GAF Chemicals Corporation.

Other operative polymeric polycarboxylates include those such as the 1:1 copolymers of maleic anhydride with ethyl acrylate, hydroxyethyl methacrylate, N-vinyl-2-pyrrolidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Additional operative polymeric polycarboxylates are disclosed in U.S. Pat. No. 4,138,477 to Gaffer and U.S. Pat. No. 4,183,914 to Gaffer et al.; each of which are incorporated by reference, and include copolymers of maleic anhydride with styrene, isobutylene or ethyl vinyl ether; polyacrylic, polyitaconic and polymaleic acids; and sulfoacrylic oligomers of M.W. as low as 1,000 available as Uniroyal ND-2.

### Thickening Agents

Thickening agents may also be incorporated into the compositions of the present invention as required. Suitable thickening agents include, but are not limited to carboxyvinyl polymers, carrageenan, hydroxyethyl cellulose, laponite and water soluble salts of cellulose ethers such as sodium carboxymethylcellulose, sodium carboxymethyl hydroxyethyl cellulose and mixtures thereof. Natural gums such as gum karaya, xanthan gum, gum Arabic gum tragacanth and mixtures thereof can also be used. Colloidal magnesium aluminum silicate or finely divided silica can be used as part of the thickening agent to further improve texture.

Useful thickening or gelling agents also include a class of homopolymers of acrylic acid crosslinked with an alkyl ether of pentaerythritol or an alkyl ether of sucrose, or carbomers. Carbomers are commercially available from B. F. Goodrich as the Carbopol® series. Certain embodiments include Carbopols include Carbopol 934, 940, 941, 956, and mixtures thereof.

Copolymers of lactide and glycolide monomers, the copolymer having the molecular weight in the range of from about 1,000 to about 120,000 (number average), are useful for delivery of actives into the periodontal pockets or around the periodontal pockets as a “subgingival gel carrier.” These polymers are described in U.S. Pat. Nos. 5,198,220, and 5,242,910, respectively both to Damani, and U.S. Pat. No. 4,443,430 to Mattei, each of which are incorporated herein by reference.

Thickening agents in an amount from about 0.1% to about 15%, optionally from about 2% to about 10%, or optionally from about 4% to about 8%, by weight of the total toothpaste or gel composition, can be used. Higher concentrations can be used for chewing gums, lozenges (including breath mints), sachets, non-abrasive gels and subgingival gels.

### Humectants

Another optional component of the topical, oral carriers of the compositions of the subject invention is a humectant. The humectant, on a pure humectant basis, generally comprises from about 0% to about 70%, optionally from about 5% to about 25%, by weight of the compositions herein. Suitable humectants for use in compositions of the subject invention include edible polyhydric alcohols such as glycerin, sorbitol, xylitol, butylene glycol, polyethylene glycol, propylene glycol, or mixtures thereof.

### Flavoring and Sweetening Agents

Flavoring agents can also be added to the compositions. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof. Flavoring agents are generally used in the compositions at levels of from about 0.001% to about 5%, by weight of the composition. Certain embodiments include an essential oil mixture of menthol, methyl salicylate, eucalyptol and thymol in view of the mixture's antimicrobial properties. A more detailed discussion on these essential oils can be found in U.S. Pat. No. 6,121,315, to Nair et al., herein incorporated by reference in its entirety.

Sweetening agents which can be used include sucrose, sucralose, glucose, saccharin (such as sodium saccharin), dextrose, levulose, lactose, mannitol, sorbitol, fructose, maltose, xylitol, saccharin salts, thaumatin, aspartame, D-tryptophan, dihydrochalcones, acesulfame and cyclamate salts (such as sodium cyclamate) and mixtures thereof. A composition preferably contains from about 0.1% to about 10% of these agents optionally from about 0.1% to about 1%, by weight of the composition.

In addition to flavoring and sweetening agents, coolants, salivating agents, warming agents, and numbing agents can be used as optional ingredients in compositions of the present invention. These agents are present in the compositions at a level of from about 0.001% to about 10%, optionally from about 0.1% to about 1%, by weight of the composition.

The coolant can be any of a wide variety of materials. Included among such materials are carboxamides, menthol, ketals, diols, and mixtures thereof. Suitable coolants include, but not limited to, the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional preferred coolants are selected from the group consisting of menthol, 3-1-menthoxypropane-1,2-diol known as TK-10 manufactured by Takasago; menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof. TK-10 is described in U.S. Pat. No. 4,459,425 to Ammo, et al., WS-3 and other agents are described in U.S. Pat. No. 4,136,163 to Watson, et al., each which patent is herein incorporated by reference.

Salivating agents of the present invention may include Jambu® manufactured by Takasago. Warming agents include capsicum and nicotinate esters, such as benzyl nicotinate. Numbing agents include benzocaine, lidocaine, clove bud oil, and ethanol. Mixtures of any of the above agent can also be used.

### Miscellaneous Carriers

Water employed in the preparation of commercially suitable oral compositions should preferably be of low ion content and free of organic impurities. Water generally comprises from about 5% to about 70%, and preferably from about 20% to about 50%, by weight of the aqueous compositions herein. These amounts of water include the free water which is added plus that which is introduced with other materials, such as with sorbitol.

The pH of the present compositions is preferably adjusted through the use of buffering agents. Buffering agents, as used herein, refer to agents that can be used to adjust the pH of the compositions to a range of about pH 4.0 to about pH 10.0. Buffering agents include benzoic acid, benzoate salt (e.g., sodium benzoate) monosodium phosphate, trisodium phosphate, sodium hydroxide, sodium carbonate, sodium acid pyrophosphate, citric acid, sodium citrate and mixtures thereof. Buffering agents can be administered at a level of from about 0.5% to about 10%, by weight of the present compositions.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its scope.

### Example 1

### A Chewing Gum Containing the Linseed Agent

The following is an example of a chewing gum composition of the present invention. The composition is formed by combining and mixing the ingredients of each column using conventional chewing gum technology.

| Ingredient | % (wt/wt) |
|---|---|
| Gum Base | 24.75 |
| 70% Sorbitol Solution | 14.75 |
| Glycerin | 6.25 |
| Sorbitol (solid) | 45.05 |
| Linseed Extract¹ | 7.00 |
| Menthol | 0.16 |
| Thymol | 0.24 |
| Methyl salicylate | 0.35 |
| Eucalyptol | 0.25 |
| Flavor | 1.20 |

¹Linseed Extract supplied by Sinclair Pharma under the trade name Salinum ®.

### Example 2

### A Spray Composition Containing the Linseed Agent

The following is an example of a spray composition of the present invention. The spray composition is formed by combining and mixing the ingredients of each column using conventional spray formulation technology.

| Ingredient | % (wt/wt) |
|---|---|
| Alcohol | 51.0 |
| Flavor | 2.8 |
| Surfactant | 2.7 |
| Sweetner | 0.7 |
| Linseed Extract | 6.0 |
| Cetylpyridinium chloride | 0.1 |
| Water | qs |

### Example 3

### A Spray Composition Containing the Tamarind Seed Agent

The following is an example of a spray composition of the present invention. The spray composition is formed by combining and mixing the ingredients of each column using conventional spray formulation technology.

| Ingredient | % (wt/wt) |
|---|---|
| Alcohol | 51.0 |
| Flavor | 2.8 |
| Surfactant | 2.7 |
| Sweetner | 0.7 |
| Tamarind Seed Extract² | 6.0 |
| Cetylpyridinium chloride | 0.1 |
| Water | qs |

²Tamarind seed extract supplied by GLYLOID ®, Dainippon Pharmaceutical Co.

### Example 4

### A Spray Composition Containing the Tamarind Seed and Linseed Agent

The following is an example of a spray composition of the present invention. The spray composition is formed by combining and mixing the ingredients of each column using conventional spray formulation technology.

| Ingredient | % (wt/wt) |
|---|---|
| Alcohol | 51.0 |
| Flavor | 2.8 |
| Surfactant | 2.7 |
| Sweetner | 0.7 |
| Tamarind Seed Extract | 3.0 |
| Linseed Extract | 3.0 |
| Cetylpyridinium chloride | 0.1 |
| Water | qs |

### Example 5

### A Mouthrinse Composition Containing the Linseed Agent

The following is an example of a mouthrinse composition of the present invention. The mouthrinse composition is formed by combining and mixing the ingredients of each column using conventional mixing and spray formulation technology.

| Ingredient | % (wt/wt) |
|---|---|
| Alcohol | 9.000 |
| Flavor | 0.820 |
| Surfactant | 0.250 |
| Benzoic Acid | 0.150 |
| Sweetner | 0.100 |
| Color | 0.005 |
| 70% Sorbitol Solution | 16.500 |
| Linseed Extract | 8.000 |
| Cetylpyridinium chloride | 0.050 |
| Water | qs |

### Example 6

### A Dentifrice Composition Containing the Linseed Agent

The following is an example of a dentifrice composition of the present invention. The dentifrice composition is formed by combining and mixing the ingredients of each column using conventional mixing and spray formulation technology.

| Ingredient | % (wt/wt) |
|---|---|
| Water | qs |
| 70% Sorbitol Solution | 40.000 |
| Sodium | 0.760 |
| Monofluorophosphate | |
| Sweetner | 1.200 |
| Sodium Phosphate (mono | 0.250 |
| basic) | |
| Sodium Phosphate (di | 0.030 |
| basic) | |
| Benzoic Acid | 0.150 |
| Color | 0.002 |
| Phosphoric Acid | 0.442 |
| Abrasive Silica³ | 15.000 |
| Thickening Silica | 2.000 |
| Titanium Dioxide | 0.350 |
| Xanthum Gum | 0.600 |
| Glycerin | 0.600 |
| Flavor | 2.300 |
| Sodium Lauryl Sulfate | 1.500 |
| Linseed Extract | 5.000 |

³Silica (abrasive and thickening) provided by W. W. Grace.

### Example 7

### An Anti-Microbial Center-Filled Lozenge with Linseed Extract and Menthol in the Center-Fill

An anti-microbial center-filled lozenge having a core containing linseed extract and menthol is prepared according to the above Method of Preparation and had a formulation as specified below.

| | | Center- | |
|---|---|---|---|
| | | Fill | |
| | Shell | % | Total |
| | % | w/w in | Product |
| | w/w in | Center- | % |
| Ingredient | Shell | Fill | w/w |
| Isomalt⁴ | 93.7316 | 0.0000 | 81.4507 |
| Purified Water | 0.6300 | 0.0000 | 0.5460 |
| Citric Acid | 0.0500 | 0.0000 | 0.0433 |
| Acesulfame | 0.0340 | 0.0000 | 0.0295 |
| Potassium Salt | | | |
| Aspartame | 0.0680 | 0.0000 | 0.0589 |
| Orange Flavor | 0.2000 | 0.0000 | 0.1733 |
| Menthol | 0.2500 | 0.2500 | 0.0000 |
| Lycasin | 0.0000 | 79.7136 | 10.6618 |
| (maltitol | | | |
| syrup), | | | |
| Roquette | | | |
| Beta Carotene | 0.0352 | 0.0352 | 0.0352 |
| 2% WD | | | |
| Emulsion, | | | |
| 3030 | | | |
| Color | 0.0012 | 0.0012 | 0.0012 |
| Linseed extract | 5.0000 | 20.0000 | 7.0000 |
| | 100.0000 | 100.0000 | 100.0000 |

⁴Hydrogenated isomalt, supplied by Palatinit of America.
The % Isomalt in the finished product refers to amount of Cooked Isomalt which will contain about 1.5% moisture.

### Method of Preparation

### Shell Preparation

The Isomalt and water are added and mixed in a suitable vessel under heating to about 165° C. to form a candy base. A suitable acid (e.g., citric acid, malic acid, tartaric acid etc.) is then added to the vessel. The candy base is then cooled to about 145° C. A suitable sweetener (e.g. a high intensity sweetener such as acesufame K, aspartame, neotame and the like or mixtures thereof) is added along with the menthol, linseed extract, flavors and the remaining ingredients.

### Center-Fill (Core) Preparation

The core material is prepared by mixing maltitol syrup (Lycasin 80/55 from Roquette America), saliva substitute or replacement agent and, if desired, a colorant in a suitable vessel under heating to form a candy base. The candy base is then cooled to about 70° C. or lower to enable the addition of a beta carotene, a suitable viscosity modifying agent, such as glycerin, sweetener (e.g. high intensity sweetener), the menthol, linseed extract, and the remaining ingredients.

The respective shell and core materials are then added to separate hoppers which materials are then combined and delivered as a stream of the respective materials to a manifold which provides for the interruptible flow of the core ingredients and a continuous flow of the shell ingredients surrounding the core. The resulting product is ejected in discrete units corresponding to the desired weight and size of the lozenge and placed in trays with individual compartments for storing the lozenge until they cool to ambient temperature.

1. An oral care composition for improved substantivity, comprising:
a. an effective amount of an oral care active; and
b. an effective amount of a substantivity enhancing agent selected from the group consisting of linseed polysaccharide base.

2. An oral care composition of claim 1 wherein the oral care active is selected from the group consisting of anti-microbial agents, desensitizing agents, tooth whitening actives, anti-stain agents, anti-tartar agents, anti-plaque agents, fluoride ion sources, tooth strengthening agents, nutrients, antioxidants, anesthetics, and mixtures thereof.

3. (canceled)

4. An oral care composition of claim 2 wherein the oral care active is a tooth whitening active.

5. An oral care composition of claim 6 wherein the tooth whitening active is peroxide.

6. An oral care composition of claim 7 wherein the peroxide is selected from the group consisting of hydrogen peroxide, calcium peroxide, carbamide peroxide, and mixtures thereof.

7-14. (canceled)

15. An oral care composition of claim 1 further comprising at least one thymol, methyl salicylate, menthol, and eucalyptol in effective amounts.

16. The oral composition of claim 1 further comprising at least one sugar alcohol.

17. The oral composition according to claim 16 wherein said sugar alcohol is selected from the group consisting of sorbital, xylitol, lactitol, mannitol, maltilol, hydrogenated starch hydrolsate, erythritol, reducing paratinose and mixtures thereof.

18. The oral composition according to claim 1 in a form selected from the group consisting of toothpaste, mouthwashes, gels, toothpowders, edible film, film forming dentifrices, chewing gums, tablets, capsules, mouth sprays and lozenges.
